(19) United States
(12) Patent Application Publication
(10) Pub. No.: US 2004/0247706 A1
(43) Pub. Date: Dec. 9, 2004

(54) Transdermal dietary supplement comprising parthenolide

(76) Inventor: Stephen C. Roberts, Minnetonka, MN (US)

Correspondence Address: KAGAN BINDER, PLLC, SUITE 200, MAPLE ISLAND BUILDING, 221 MAIN STREET NORTH, STILLWATER, MN, 55082, US

(21) Appl. No.: 10/457,038

(22) Filed: Jun. 6, 2003

(51) Int. Cl. A61K 35/78; A61K 9/70; A61K 31/365

(52) U.S. Cl. 424/729; 424/756; 424/764; 514/473

(57) ABSTRACT

An improved method for administering parthenolide and/or feverfew extract as a dietary supplement is provided, wherein a dietary supplemental amount of parthenolide in a predetermined dosage amount of at least about 1 mg of parthenolide is transdermally administered to a patient. Transdermal delivery systems are also provided.

### FIELD OF THE INVENTION

This invention relates to the field of natural dietary supplements. It is directed particularly towards the supplementation of diets, and most particularly towards supplementation of the diet with parthenolide.

### BACKGROUND OF THE INVENTION

Dietary supplements are materials provided to animals, typically multi-vitamin and mineral supplements, for treating specific medical conditions and as general nutritional supplements to promote and maintain good health of animals, particularly mammals. In particular, compositions and methods for optimizing the general health of both men and women by supplementing the daily diet with specific and multi-vitamin compositions. Dietary supplements are typically administered for ready consumption by the user, either as a combination with food or in a delivery vehicle suitable for swallowing by the user.

U.S. Pat. No. 6,475,511 to Gohlke, et al. discloses a dietary supplement for mammalian consumption, and particularly human consumption, for the purpose of stimulating the immune system, inhibiting infection and increasing tissue repair and healing. Comprising colostrum, lactoferrin, and with modified citrus pectin as an optional component, the dietary supplement is administered in ‘mucosal delivery format’: a dosage form that promotes effective absorption through the lining of the oral cavity.

U.S. Pat. No. 6,319,510 to Yates discloses a gum pad for the topical or systemic delivery of a wide range of pharmaceutical and nutritional agents. The gum pad is a laminate composed of: (a) a synthetic base or backing layer which is configured to be held in place on the gingiva (gums) in the mouth; (b) an intermediate, reservoir layer for containing medication therein; and (c) a semi-permeable outer layer facing outwardly toward oral mucosal tissues in the mouth which will allow saliva to enter and dissolve the medication in the reservoir layer into solution and pass the diffused saliva-medication solution outwardly to the oral mucosal tissues. The backing layer is placed on the gum so that the semi-permeable outer layer faces outwardly toward the buccal mucosa. Saliva enters the semi-permeable layer and dissolves the medication in the reservoir layer, then diffuses outwardly through the semi-permeable layer to the mucosal tissues in the mouth where it is readily absorbed into the circulatory system. Plants or plant components are also described as being capable of being delivered by the gum pad, “including those from garlic, ginkgo biloba, kava kava, noni, ginseng, saw palmetto, milk thistle, stinging nettle, eucalyptus, aloe vera, feverfew, nasturtium, Ma Huang, and echinacea.”

Feverfew is an herb that is widely available and has been investigated in modern times. Historically, feverfew is known to have been used in the treatment of fevers, from whence it derives its name, and also in rheumatic conditions. Feverfew is used in homeopathic remedies, but homeopathy recognizes no role for feverfew in the treatment of headaches. An authoritative homeopathic text is “A Dictionary Of Practical Materia Medica” by John Henry Clarke, M.D., recognized as such by the United States Food and Drug Administration, (see the “Compliance Policy Guide: Conditions Under Which Homeopathic Drugs may be Marketed” http://www.fda.gov/ora/compliance ref/cpg/cpgdrg/cpg 400-400.html). The Clarke text gives no indication for the use of feverfew in the treatment of headaches. This same text defines the appropriate preparation of feverfew as “a tincture of fresh leaves.” A tincture is a concentrated herbal extract prepared by soaking an herb in alcohol for an extended period of time. The result is an alcoholic extract referred to by homeopathic practitioners as the “mother tincture.” This “mother tincture” is then subject to numerous serial dilutions with the resulting homeopathic drug being extremely dilute. Classic homeopathic remedies do not rely on any effect from the substance first contained in the starting material (“mother tincture” in this case) and often statistically contain virtually no actual molecules of the original substance (feverfew). Instead, these remedies rely on the “imprint” or “energy” of the original substance to exert an effect. Consistent with the precepts of homeopathy, the remedy thus prepared is felt to become increasingly potent, indeed stronger and more effective, as it becomes more and more dilute. Some of these homeopathic remedies may have been administered sublingually, or may yet be administered sublingually by those presently adherent to the practice. Such purported remedies would certainly contain less than 0.01 mg/ml of parthenolide. There is little to no clinical support for any of the multitude of homeopathic remedies. Thus it is not surprising but still noteworthy that no clinical evidence exists for the effective use of feverfew in homeopathic medicine as a treatment for headache, especially as homeopathy does not recognize nor endorse this use. The amounts of supplement remaining in the homeopathic product, if not zero, are still substantially less than would be considered needful for general and beneficial supplementation of the diet.

Herbal medicine, as a field distinct from classical homeopathy, has in fact recognized the potential value of feverfew in the prophylactic (preventative) treatment of migraine. Fresh feverfew leaves have sometimes been chewed by subjects wishing to rid themselves of migraine. However, a common adverse effect reported by those who have used this technique is the generation sores in the mouth and sensitization of oral tissues. Additionally, many patients find this mode of administration to be crude and unpleasant.

In addition to raw leaves, feverfew tablets or capsules have been and are employed by practitioners of herbal medicine. These are widely available in any “health food store” for purchase by the general public. The PDR for Herbal Medicines lists migraine, arthritis, rheumatic diseases and allergies as the indications for feverfew usage (PDR for Herbal Medicines, Thompson Medical Economics, Second Edition, Feverfew, 306-309, 2000.) Several studies published in leading medical journals, including “Lancet” (Murphy, J J Lancet 1988 Jul. 23;2(8604): 189-92), and “The British Medical Journal” (Johnson, E S British Medical Journal 1985 Aug. 31; 291(6495):569-73), have suggested a potential role for feverfew in reducing the incidence and/or severity of migraines. The Murphy study administered one capsule of feverfew leaves to be swallowed by the patient, wherein each capsule contained about 2.19 micromoles of parthenolide (about 0.5 mg). The Johnson study administered two capsules of freeze dried feverfew powder every morning. The daily dose was therefore 50 mg feverfew. The parthenolide content of this feverfew powder was not reported. Patients reported a reduction in the number and/or severity of migraine attacks, with no side effects reported by either study. Most recently however, several systemic reviews of feverfew use in the prevention of migraine have been published (Vogler BK, “Feverfew as a preventive treatment for migraine: a systematic review.” Cephalalgia 1998 Dec.;18(10):704-8) and (Pittler M H, “Feverfew for preventing migraine.” Cochrane Database Syst Rev 2000;(3):CD002286) both of which reviews concluded that the efficacy of feverfew for the prevention of migraine “has not been established beyond reasonable doubt.” Pittler also noted that “the trial with the highest methodological quality, which was also among the largest, found no significant difference between feverfew and placebo.” Most clinicians in the United States do not consider feverfew an effective prophylactic treatment for migraine and as such do not endorse its use. Feverfew has not been used for the acute relief of migraine attacks. Each of the studies investigated only its prophylactic use. The use of feverfew acutely, for relief of headaches once they have begun, has not been studied and there is no scientific literature that directly suggests that it might be effective. In addition, the prophylactic effect is not said to be noticeable for some number of weeks (2-12) after having first initiated use of feverfew, regardless of the form of feverfew employed (tablets, leaves, etc.). Recommended dosages of feverfew tablets or capsules are 200 to 250 mg one to three times daily, there being no suggestion that alternate routes of administration might prove beneficial.

A product currently available on the market is sold under the name MigraSprays®, which is stated to be a patented over the counter homeopathic drug intended to be a comprehensive approach for the treatment and prevention of migraine headaches. MigraSpray contains the active ingredients feverfew, polyporus, goldenseal and dandelion. MigraSpray is sprayed under the tongue (sublingual administration), which promotes enhanced bioavailability and rapid absorption by directly entering the bloodstream through the mucous membrane avoiding degradation from exposure to the gastrointestinal tract and liver. Product literature claims that this sublingual delivery system allows MigraSpray to provide rapid relief from migraine headache pain and other associated symptoms in an average of less than 7 minutes. However, this product, like other products sold as “homeopathic” treatments, delivers an extremely low dosage of feverfew. The amount of parthenolide reported to be present in this composition is 0.0112 mg/dose.

Additionally, it is noteworthy that the content of parthenolide in feverfew may vary to a great extent depending on the particular variety of Tanacetum parthenium plant grown, and also the manner of processing the feverfew herb. Parthenolide has been found to be unstable and sensitive to processing. Thus, the collection and processing steps carried out incorporating feverfew into a product may reduce or destroy the parthenolide content of the feverfew. Without a standardization of the parthenolide content of feverfew used in the process and careful control of the manufacturing process, great inconsistency is observed in parthenolide content from batch to batch of product.

U.S. Pat. No. 5,503,843 to Santus discloses a transdermal patch for the delivery of a specific Compound to the skin of a patient. The patch comprises a backing layer, a drug depot comprising the compound and a permeation enhancer composition.

U.S. Pat. No. 5,837,289 to Grasela, et al. discloses a composition and procedures for its formation and administration to provide a convenient, efficacious and simple transdermal administration of medications from a topically applied cream. The composition incorporates at least two separate penetration enhancers which function synergistically to provide for rapid but controllable transport of the medication from the cream into the skin. The use of a plurality of penetration enhancers, at least one of which facilitates the separation of medication from the cream and at least a second of which alters the structure of the outer layers of skin, particularly the stratum corneum, enhances migration of the drug through the stratum corneum.

U.S. Pat. No. 6,410,062 to Callaghan, et al. describes a method of treating and preventing inflammatory disorders and related conditions by applying a topical composition comprising an effective amount of an extract of feverfew, where the extract is substantially free of α-unsaturated γ-lactone, and particularly substantially free of parthenolide.

U.S. Pat. No. 5,905,089 to Hwang, et al. describes the use of sesquiterpene lactones for treatment of severe inflammatory disorders; possible route of administration is transdermal. The term “transdermal” in this patent is described in a very specific manner that apparently does not contemplate topical application of a composition or a patch, stating that the composition may be “administered transdermally, for example in the form of a slow-release subcutaneous implant.” See column 6, line 26.

### SUMMARY OF THE INVENTION

The present invention provides an improved method for administering parthenolide and/or feverfew extract as a dietary supplement comprising transdermally administering a dietary supplemental amount of parthenolide in a predetermined dosage amount of at least about 1 mg of parthenolide, and more preferably from about 1 mg to about 50 mg of parthenolide.

Various systems, including compositions and composites, are disclosed herein for transdermal delivery of parthenolide to a patient. It is contemplated that such compositions and composites may contain an amount of parthenolide that exceeds the above indicated administration amounts. This is necessary because it is axiomatic that not all parthenolide present in any given matrix will be delivered to the patient. Thus, some parthenolide may be retained in the composition or composite at the time of removal from the patient. For purposes of the present invention, when an amount of parthenolide is indicated to be “administered,” it is contemplated that the transdermal delivery system is designed to delivery that amount to the patient during the stated time period, and is not intended as an indication of the total amount of parthenolide that is present in the matrix of the transdermal delivery system. In a preferred embodiment, a transdermal delivery system is provided wherein parthenolide is administered in a total transdermally administered amount of from about 1 mg to about 50 mg of parthenolide within a two hour period. In another preferred embodiment, a transdermal delivery system is provided wherein parthenolide is administered in a total transdermally administered amount of from about 1 mg to about 50 mg of parthenolide within a 24 hour period.

In one aspect of the present invention, convenient systems for administration of parthenolide, and as another embodiment, feverfew extract containing parthenolide, are provided wherein compositions are provided as a transdermal patch for transdermal administration. Compositions and delivery systems of the invention that are preferred also render the dietary supplement of the invention particularly adaptable to self-monitored dosages, and are especially appropriate for regimes of self administration.

### BRIEF DESCRIPTION OF THE DRAWINGS

FIG. 1 shows a cross section of a simple adhesive patch having an impermeable backing layer, a parthenolide containing adhesive layer, and a release liner.

FIG. 2 shows a cross section of a matrix transdermal patch having an impermeable backing layer, a parthenolide source comprising a polymer matrix in which parthenolide is dispersed, a peripheral adhesive layer, a release liner. The patch may also contain a porous membrane.

FIG. 3 shows a cross section of a reservoir type transdermal patch having an impermeable backing, a parthenolide source, a porous membrane, an adhesive layer, and a release liner. The backing layer and the membrane are sealed at their peripheral edges, thus defining a parthenolide source.

### DETEILED DESCRIPTION

Previous modes of administration of parthenolide have generally administered parthenolide and/or feverfew through the GI tract, where it is believed to be rapidly degraded by the acidic and enzymatic conditions of the stomach and intestine. Other administration attempts have utilized far too small an amount of parthenolide to be effective as a dietary supplement. The practice of chewing feverfew leaves suffers from a number of disadvantages, including widely varied parthenolide content in any particular quantity of feverfew leaves. Because the amount of parthenolide in leaves cannot be standardized among a sufficient amount of leaves, it is not possible for a known quantity of parthenolide to be administered in this manner. Further, studies have shown that in some cases, depending on various production factors, samples of feverfew leaves have been found to contain no parthenolide whatsoever.

Compositions of the present invention comprise a dietary supplemental amount of parthenolide that is in a predetermined dosage amount of at least about 1 mg of parthenolide. Because the dosage is predetermined, the user can rely on a standard and reproducible quantity of the desired active to be administered. Thus supplemental parthenolide can therefore be provided in a form that provides ready self monitoring and control of treatment regimes. Preferably, the composition comprises from about 1 to about 50 mg, and more preferably from about 1 to about 30 mg of parthenolide in each dosage unit. Preferably, these amounts of parthenolide are delivered at a rate such that the amount is administered over a 2 hour period or a 24 hour period.

When an active agent is administered intravenously, intramuscularly, orally or mucosally, the initial level of the active agent in the blood rapidly rises to a maximum, which is generally much higher than the therapeutically effective level of the active agent. Sometimes initial levels of actives administered orally may reach toxic concentrations resulting in undesirable side-effects. This is known as “overdosing.” After the maximum level in the blood is reached, the concentration then falls slowly as the active is distributed, metabolized, excreted, or degraded. Eventually, the blood concentration of the active agent falls below the therapeutically effective level (i.e., there is “underdosing”). At this point, the active agent needs to be re-administered to achieve effectiveness. Maintaining the blood concentration of the active agent between the minimum therapeutically effective level and toxic levels is important. One way to achieve this is to administer lower active agent doses to the patient more frequently. This, however, is an unacceptable alternative in most instances, due to problems with patient compliance.

Transdermal delivery of parthenolide offers a means of circumventing the problems of overdosing and underdosing that may be associated with conventional parthenolide delivery methods. The transdermal delivery of parthenolide can be designed so that the rate of delivery of the parthenolide closely follows the rate of the clearance of the parthenolide from the environment, thus keeping constant levels of parthenolide in the blood, and reducing parthenolide waste and overdosing problems.

The term “pharmaceutically acceptable” or “therapeutically acceptable” refers to a substance which does not interfere with the effectiveness or the biological activity of the active ingredients and which is not toxic to the host or patient. The term “patient” refers to a mammal that is being treated. Preferably the patient is a human.

Parthenolide is a sesquiterpene lactone that may be obtained from a number of sources. A preferred source of parthenolide is by extraction from the feverfew plant (Tanaecetum parthenium), which is also known, for example, as Chrysanthemum parthenium, Chrisanthemum parthenium, Pyrethrum parthenium, Tanacete parthenii herba or folium, Matricaria parthenoides, Matricaria parthenium, Leucanthemum parthenium, Matricaria parthenium, Spanish pellitory, Featherfew, Featherfoil, feather-fully, and by a number of common names, various of which are used throughout the world (Midsummer daisy, Bachelor's buttons, Altamisa, nosebleed, flirtwort, ague plant, devil daisy, feddygen fenyw (Welsh), maid's weed, Missouri snakeroot, mutterkaut (German), prairie-dock, vetter-voo, wild chamomile, grande camomille (French), Santa Maria (Spain), febrifuge plant.) The extract may be obtained by techniques known in the art using solvents such as petroleum spirits or polar organic solvents. See U.S. Pat. No. 5,384,121 to Rhodes, and also WO 94 06800; EP 0 553 658; WO 92 11857; GB 2,166,952; EP 98 041; WO 98 39018.

Compositions of the present invention may comprise additional ingredients providing nutritional benefit to the ultimate composition. Thus, additional ingredients such as sesquiterpene lactones, vitamins, mineral or other ingredients desirable for supplementing the dietary needs and/or imparting healthful benefits to the patient in need thereof are contemplated.

The extract of the feverfew plant contains parthenolide, and may additionally contain other components such as Polyynes, Flavonoids and Volatile oils including camphor, bomeol and others, each of which may contribute as a dietary supplement benefits of the composition disclosed herein. Feverfew also contains relatively large quantities of sesquiterpene lactones.

In addition to parthenolide, feverfew is known to contain the following non-ubiquitous chemicals: 1-Beta-hydroxyarbusculin, 10-Epicanin, 8-Beta-reynosin, Apigenin-7-glucoside, Chrysanthemolide, Chrysanthemonin, Chrysartemin-A, Chrysartemin-B, Cosmosiin, L-Bomeol, L-camphor, Mangoliolide, Reynosin, Santamarin, Tanaparthin, Tanaparthin-1-alpha, 4-alpha-epoxide, Tanaparthin-1-beta,4-beta-epoxide, tenetin 3-b-hydroxyparthenolide, seco-tanaparthenolide A, canin, artecanin, and balchanin.

Because feverfew extract may contain additional beneficial components, compositions comprising the extract of feverfew are generally preferred for use in the present invention as compared to compositions comprising a highly purified parthenolide that has been isolated from the additional components naturally occurring in feverfew extract. Preferred embodiments of the present invention use feverfew extract that has been standardized to contain a predetermined standardized parthenolide concentration of preferably not less than about 1.0%, and more preferably from about 1.2% to about 10%. Higher concentration parthenolide compositions may become readily available, which may advantageously reduce the amount of liquid required in the composition for delivery of the active to the user. While the source of parthenolide in compositions of the present invention is preferably feverfew as discussed above, it may alternatively be obtained from any number of other plant species, where it generally occurs in substantially lower concentrations. Such plant species include especially other members of the Compositae family, which include especially the many species of chrysanthemums, daisies, marigolds, chamomile, yarrow and aster. Parthenolide can also be obtained from tansy. Alternatively, parthenolide may be made by any appropriate synthetic route.

The composition to be used in the present invention may optionally comprise additional active ingredients. These active ingredients may also be provided as a dietary supplement or may provide other physical benefits, provided that the benefit of parthenolide is not adversely affected. In one aspect, preferably additional amounts of already present sesquiterpene lactones or additional sesquiterpene lactones are incorporated in the compositions of the present invention. Preferred such sesquiterpene lactones include especially those which are known to be contained in (naturally occur in) feverfew, such as 3-Beta-hydroxyparthenolide, seco-tanaparthenolide A, canin, artecanin, chrysanthemonin, chrysartemin A and B, santamarin and balchanin, as well as those occurring in other plant species such as encelin, leucanthin B, enhydrin, melampodin A, tenulin, confertiflorin, burrodin, psilostachyin A, costunolide, guaianolide, cinerenin, artemisinin, aristolactone, lactarorufin A, bilobalide, helenalin, furandiol. Sesquiterpene lactones in addition to parthenolide may be isolated from plants such as dandelion, burdock, butterburr, mugwort and sunflower plants, among others.

Compositions to be used in the present invention may optionally additionally comprise other naturally occurring components and extracts, including those as identified in the HPUS. Preferred additional components are extracts indicated for use in treatment of headaches, inflammation, nausea or anxiety. Particularly preferred additional components are the extracts of bay leaf and/or ginger and/or green tea, or the isolated components thereof. A particularly preferred isolated component of green tea is L-theanine.

Particularly preferred compositions of the present invention contain substantially no active ingredients other than those that are extractable from herbal sources. In a particularly preferred embodiment of the present invention, the compositions contain substantially no active ingredients other than those that are extractable from feverfew, ginger and green tea sources. In another particularly preferred embodiment, the compositions contain substantially no active ingredients other than those that are extractable from feverfew and ginger. It has surprisingly been discovered that the use of only active ingredients that are extracted from herbs provide particular benefit to the user in being both effective in the treatment of migrainous headache, and also providing natural healing conditions particularly suited to the well being of patients. Surprisingly, these natural ingredients have been found to be effective in the indicated dosage ranges when administered in a sublingual regimen as described herein. Such compositions contain parthenolide in the amounts as discussed earlier, and preferably contain less than about 400 mg of any given natural active ingredient per dose.

In a particularly preferred embodiment, the composition to be used in the present invention additionally comprises ginger extract at a total administered amount preferably not exceeding about 400 mg, and more preferably not exceeding about 250 mg of ginger extract. Particularly preferred compositions comprise ginger extract as about 0.1-10% of the total composition.

In another particularly preferred embodiment, the liquid composition additionally comprises L-theanine, either as an isolated component or as a constituent of green tea extract, but in either case at a total administered amount preferably not exceeding about 400 mg of L-theanine. Particularly preferred compositions comprise L-theanine as about 0.1-10% of the total composition.

In a particularly preferred embodiment of the present invention, the compositions of the present invention are provided at a pH of from about 2.0 to about 6.5, more preferably at a pH of from about 2.5 to about 6.0, and more preferably at a pH of from about 3 to about 5. Surprisingly, it has been found that compositions having a pH of the range indicated above are much more effective than compositions outside of the indicated range. Various pH adjusters may be used to adjust the pH of the composition to the desired level. Examples of suitable pH adjusters include hydrochloric acid, citric acid, phosphoric acid, acetic acid, tartaric acid, sodium hydroxide, potassium hydroxide, sodium carbonate, sodium hydrogen carbonate, boric acid, sodium borate, and the like. Preferably, the pH of the composition is adjusted to be acidic using ascorbic acid.

Preferably, the composition is buffered by a pharmaceutically acceptable buffer. Examples of buffering agents include borate buffers, citrate buffers, phosphate buffers, tartarate buffers, acetate buffers, carbonate buffers, and amino acid salts, etc. Most preferably, the buffer is sodium citrate.

Preferably, the composition comprises 1%-10% feverfew extract, 0.1-10% ginger extract, 0.1-10% L-theanine, and 10-98% water. Preferably, the feverfew extract has a standardized parthenolide concentration of not less than about 1.0%, and more preferably from about 1.2% to about 10%.

In addition to the advantage of being able to control the delivery rate of parthenolide, transdermal delivery also provides a comfortable, convenient and non-invasive method of administering parthenolide. Gastrointestinal irritation and other side- effects associated with oral parthenolide delivery may be reduced or eliminated, and patient anxiety regarding invasive delivery methods, such as needles, is also eliminated.

Further, the transdermal administration avoids the “first pass effect,” which often results when a medication is administered orally and thus has to pass through various organs, including the liver, before reaching the affected area of the body. These organs can absorb or chemically alter significant quantities of the passing medication, thus requiring that large excess quantities of the medication by administered initially to insure that an effective quantity of the medication will ultimately reach the affected area of the body.

In one embodiment of the present invention, compositions are provided in the form of a cream or other spreadable or moldable material. Using these formulations, parthenolide may be effectively topically administered by application of the cream to many bodily areas where a patch either will not fit or cannot be shaped to conform to the skin contours. Such compositions may be readily prepared by the routineer in the field, by consulting with established formularies and substituting the indicated active ingredients as taught herein.

For example, an appropriate cream composition may be formulated by forming an organogel from lecithin and isopropyl palmitate, as disclosed in U.S. Pat. No. 5,837,289. Suitable gel structures thus may be formed and used as the base for a cream composition. Parthenolide is preferably solubilized with a solvent, such as water, alcohol or other appropriate solvent, and mixed into the matrix to formulate an appropriate composition for application to the dermis of a patient, for transdermal delivery of parthenolide to the patient.

Additional components, such as cosmetic agents, binders, thickeners, preservatives and fragrances may be present in the topically applied cream or other moldable or spreadable material.

Cosmetic agents which may be used in the compositions of this invention may include, but are not limited to those agents which prevent potential skin irritation, such as emollients, vitamins and antioxidants (e.g., vitamin E) and herbal extracts (e.g., aloe vera). Further, the cosmetic agents may include humectants, antioxidants/preservatives, plant extracts, surface active agents, and the like. Examples of humectants include glycerol, sorbitol, propylene glycol, ethylene glycol, 1,3-butylene glycol, polypropylene glycol, xylitol, maltitol, lactitol, oat protein, allantoin, acetamine MEA, hyaluronic acid and the like. They may be used either singly or in combination.

Binders or thickeners may be used in the compositions of this invention to provide substantivity and physical stability to the compositions. Binders or thickeners suitable for use in the compositions of this invention include cellulose derivatives such as alkali metal salts of carboxymethylcellulose, methyl cellulose, hydroxyethyl cellulose and sodium carboxymethylhydroxyethyl cellulose, alkali metal alginates such as sodium alginate, propylene glycol alginate, gums such as carrageenan, xanthan gum, tragacanth gum, caraya gum and gum arabic, and synthetic binders such as polyvinyl alcohol, polysodium acrylate and polyvinyl pyrrolidone. Thickeners such as natural gums and synthetic polymers, as well as coloring agents and fragrances also are commonly included in such compositions.

Examples of preservatives which may be used in the compositions of this invention include, but are not limited to, salicylic acid, chlorhexidine hydrochloride, phenoxyethanol, sodium benzoate, methyl para-hydroxybenzoate, ethyl para-hydroxybenzoate, propyl para-hydroxybenzoate, butyl parahydroxybenzoate and the like.

Examples of fragrances which may be used in the compositions of this invention include menthol, anethole, carvone, eugenol, limonene, ocimene, n-decylalcohol, citronellol, a-terpineol, methyl salicylate, methyl acetate, citronellyl acetate, cineole, linalool, ethyl linalool, vanillin, thymol, spearmint oil, peppermint oil, lemon oil, orange oil, sage oil, rosemary oil, cinnamon oil, pimento oil, cinnamon leaf oil, perilla oil, wintergreen oil, clove oil, eucalyptus oil and the like.

The compositions of the present invention may be prepared in a number of forms for topical application to a patient. For example, the composition may be applied in a gel, cream, ointment, shampoo, scalp conditioners, liquid, spray liquid, paint-/brush-on preparation or aerosol. In addition the composition may be impregnated on a bandages, hydrocolloid dressing, treatment patch or on cloth wipe products, such as baby wipes or facial wipes.

The compositions of this invention may be in the form of emulsions, such as creams, lotions and the like. Such compositions may have more than one phase and may include surface active agents which enable multiphase emulsions to be manufactured.

Optionally, a cover sheet may be applied over the applied cream or spreadable material containing parthenolide, and further the cover sheet may be secured with an adhesive sheet or strip to protect against undesired exposure of the applied cream to the elements.

Preferably, transdermal application of Active is carried out by use of a transdermal composite, commonly referred to as a “patch.” In this embodiment, the transdermal composite is a preconstructed composite capable of adhering to the dermis of a patient, having an effective amount of Active that can be delivered from the transdermal composite, and a protective overlay material that substantially prevents undesired loss of Active to the air or to surfaces that may contact the outer portion of the transdermal composite.

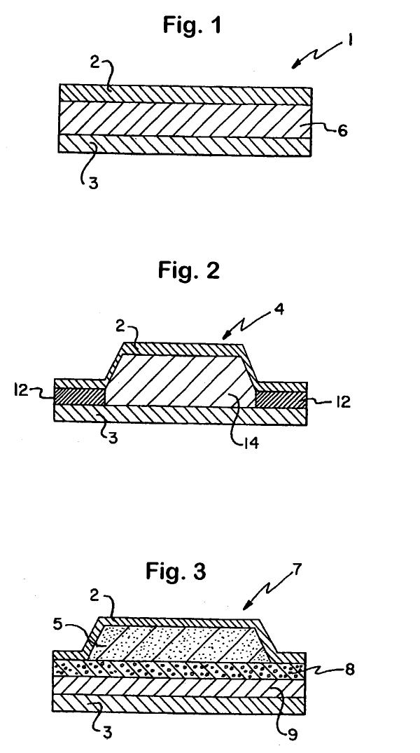

The transdermal composite may be provided in a number of configurations, as exemplified in FIGS. 1 through 3.

In one embodiment of the invention, the transdermal patch for the delivery of parthenolide (referred to as the Active) is a simple adhesive patch, as shown in FIG. 1. In FIG. 1, the patch 1 comprises an impermeable backing layer 2, a release liner 3, and an Active/adhesive containing matrix 6. The impermeable backing layer 2 defines the top of the delivery device, i.e., the side furthest away from the skin when the device is in use. The backing forms an occlusive layer that prevents the loss of Active and/or enhancers to the environment and protects the patch from contamination from the environment. The backing layer may be opaque so as to protect the Active from light.

The backing layer can be made from standard commercially available films for medical use, such as those supplied by 3M Corporation, St. Paul, Minn.; Dow Chemical, Midland, Mich.; or AF Packaging, Winston-Salem, N.C. Suitable materials which can be used to form the backing layer include films or sheets of polyolefin, polyester, polyurethane, polyvinyl alcohol, polyvinylidene, polyamide, ethylene-vinylacetate copolymer, ethylene-ethylacrylate copolymer, and the like, metal-vapor deposited films or sheets thereof, rubber sheets or films, expanded synthetic resin sheets or films, unwoven fabrics, fabrics, knitted fabrics, paper, and foils. These materials can be used individually or as laminates. These films can be pigmented or metalized.

In some aspects of the invention, the patch may include a peel strip or release liner 3 to cover the surface of the pressure-sensitive adhesive during storage, and prevent evaporative loss of the Active or enhancer(s). The release liner may be formed with dimples for decreasing contacting surface with the adhesive layer, and it may also be formed with a pull-tab for making it easier for removing it from the device.

The peel strip may be made from any impermeable film, such as is specified for the backing layer. Additionally it may be made from metal foil, Mylarm™ film, polyethylene terephthalate, or any material normally used for this purpose in the art that is compatible with the Active and the chosen adhesive. Examples of suitable compositions for the release liner include siliconized polyester, poly (1,1-dihydroperfluoroctylmethacrylate), fumed silica in silicone rubber, end-capped siliconized polyethylene terephthalate, polytetrafluoroethylene, cellophane, a film of polyvinyl chloride having titanium dioxide dispersed therein, and the like.

In the simple adhesive matrix patch of FIG. 1, the Active source layer is comprised of the Active and an adhesive, the layer attaching directly to the skin of the patient after the peel strip or release liner is removed. In the preferred embodiments of the invention, the Active source layer also comprises one or more enhancers.

Generally, the selection of the adhesive is important to the proper functioning of the transdernal delivery device. This is particularly true if a plasticizer-type enhancer is placed in the adhesive layer. Specifically, the adhesive layer must retain its functioning properties in the presence of the plasticizer-type and solvent-type enhancers, as well as upon exposure to the Active.

Alternatively, the adhesive may comprise matrix regions interspersed throughout the adhesive, wherein the matrix regions comprise Active. These matrix regions act as small reservoirs from which Active is released.

FIG. 2 shows a matrix patch, an alternative embodiment of the transdermal patch of the invention. In FIG. 2, the patch 4 comprises an impermeable backing layer 2, a release liner 3, a matrix layer 14 comprising a matrix in which Active is dispersed, and a peripheral adhesive layer 12. The matrix may be a polymer matrix, or a gel or cream in which the Active resides. In some embodiments, the patch may also include an optional porous membrane layer. In yet other embodiments, the patch may have an adhesive layer that is co-extensive with the skin facing surface of the patch. The matrix layer 14 comprises the Active, and one or more enhancers dispersed in a polymeric matrix.

The matrix layer may also comprise additional components such as diluents, stabilizers, vehicles, biocides, antioxidants, anti-irritants and the like. A preferred embodiment of the matrix patch is a matrix patch with a peripheral adhesive annular ring and an Active source having a hydrogel matrix or a foam matrix.

A further embodiment of the invention is the reservoir type patch which allows a higher loading level of active material, and usually, a higher loading level of enhancer. An example of the reservoir type patch is shown in FIG. 3. The patch 7 is comprised of an impermeable backing layer 2 which is sealed at its periphery to an inert membrane 8, thereby defining between these two layers an Active source 5. An adhesive layer 9 is affixed to the skin facing side of the patch. The patch also comprises a release liner 3. The Active source contains the Active, and optionally one or more enhancers or gelling components. Thus, in the reservoir type patches, a membrane separates the Active reservoir from the adhesive layer. In some embodiments the membrane is a non-rate controlling membrane. According to the present invention, a non-rate controlling membrane is one in which the rate of permeation of the enhancer(s) and Active through the membrane is greater than their permeation rate through the skin or any other portion of the device (typically two to five times greater or more). Thus, a non-rate controlling membrane is extremely permeable to the enhancer(s) and the Active contained in the reservoir.

In other embodiments, the membrane may be a rate-controlling membrane. As used herein, a rate-controlling membrane is one in which the rate of permeation of the enhancer(s) and the Active through the membrane is less than or equal to their permeation rate through the skin or any other portion of the device. Rate-controlling membranes are described, for example, in U.S. Pat. Nos. 4,460,372 and 4,379,454. The membrane may comprise a microporous or porous material. Microporous membranes have a distinct pore structure with pores ranging in diameter from approximately 0.08 to 0.5 microns, preferably from about 0.1 and 0.4 microns, and more preferably from about 0.2 and 0.4 microns. Examples of suitable microporous membranes include polyethylene and polypropylene films, nylon, and nitrocellulose film.

In the reservoir type patches, the membrane and the backing layer are sealed at their peripheral edges to form the Active reservoir. This seal should be substantially fluid-tight to prevent Active leakage from the reservoir through the seal between the backing layer and the membrane. As used herein, the term “peripheral edges” of the membrane and backing layers refer to the areas that are sealed together to define the Active reservoir. Therefore, extraneous membrane and backing layer material may extend outwardly from the Active reservoir and peripheral edge.

The Active reservoir contains a solution, suspension, or gel of the Active and the permeation enhancers, as well as diluents, such as water, and vehicles or other additives. The Active can be dispersed in the solution, suspension, or gel in either a dissolved or undissolved state.

A gelling agent may be incorporated into the reservoir or matrix to increase the viscosity and rheological characteristics of the Active and enhancers. The gelling agent comprises a pharmaceutically-acceptable material that is capable of increasing viscosity of the reservoir solution. Typically, the Active delivery devices described herein will employ cellulosic materials as the gelling agent. Examples of suitable cellulosic materials include cellulose, cellulose derivatives, alkylcellulose, hydroxy-(lower alkyl) cellulose derivatives where the alkyl group contains one to six carbons, carboxyalkylcellulose and the like. Other gelling agents include PVP, CMC, Klucel, alginates, kaolinate, bentonite, or montmorillonite, other clay fillers, stearates, silicon dioxide particles, carboxy polymethylene, ethylene maleic anhydride, polyacrylamide, and poly (methyl vinyl ether maleic anhydride.)

The reservoir or matrix layer also may include diluents, stabilizers, vehicles, biocides, antioxidants, anti-irritants and the like. For example, water is frequently utilized as a diluent in the reservoir type patches. Typically water will be present in the reservoir in an amount not greater than about 50 wt %, based on the reservoir fill solution; preferably, not greater than 40 wt %. Other diluents which will frequently find use in the Active delivery devices described herein include glycerine and propylene glycol.

A pressure-sensitive adhesive layer is affixed to the membrane opposite to the backing layer. In one embodiment, the adhesive layer should interact minimally with the Active. In another embodiment, the adhesive may comprise Active for additional delivery of Active to the user. The adhesive should adhere firmly to the membrane, but removably to the release liner. The device should stick securely to the wearer for extended periods, yet be removed at the desired time with minimum discomfort. The device should not give rise to undue skin irritation, allergic reactions or other dermatological problems. These properties must be maintained from the time of patch manufacture, throughout storage, and up to and throughout the time of application.

An alternative embodiment of the reservoir patch has a peripheral adhesive, wherein the area of the adhesive layer is not co-extensive with the active releasing area of the patch, but rather forms an annular ring around the active releasing area of the patch. The delivery of the Active thus is not primarily through the adhesive layer of the patch, although some lateral diffusion may occur within the patch, resulting in delivery of active substance through the adhesive at the periphery of the patch. The shape of the peripheral adhesive region will vary with the shape of the patch, but will generally comprise the outer perimeter of the patch, in order that an adequate adhesive seal is maintained between the skin and the patch to prevent the patch from falling off. The percentage of the patch that comprises the peripheral adhesive portion depends on the type of adhesive, the type of backing layer, the length of time the patch will be worn, and the weight and loading of Active in the patch. Such determinations will be apparent to the skilled artisan.

Prior to use, the patches typically are stored in laminate foil pouches, both to prevent contamination and to avoid Active and/or enhancer(s) loss. Such pouches are standard in the industry, and therefore may be selected by the routineer in this art. The patch may be assembled by any of the techniques known in the art for producing transdermal patches. The patches may be of various shapes, but the round shape is preferred as it contains no corners and thus is less easily detached from the skin.

For adequate skin penetration of most Actives, a chemical permeation enhancer may be desired. As used herein, the term “enhancer” is meant to encompass any enhancer or combination of enhancers that increases the flux of a substance across a mammalian stratum corneum. There are numerous possible permeation enhancers that can be used and they are typically categorized into two groups, solvent-type enhancers and plasticizing-type enhancers.

Plasticizer-type enhancers refers to fatty acids, fatty acid esters, fatty alcohols and similar hydrophobic compounds that are capable of increasing the permeability of Actives to the stratum corneum. Without limiting the scope of the present invention, the following is proposed as the mechanism of action of the plasticizer-type enhancers. It is believed that the function of the plasticizer-type enhancers is to migrate into the upper stratum corneum layers of the skin and disrupt the lipids which occupy the extracellular spaces of the stratum corneum. The stratum corneum layer, although only 25-50 microns thick, is the principal barrier to transdermal permeation. The plasticizer-type enhancers that migrate into the skin serve to increase the mobility and diffusion of the Active into the skin.

Plasticizer-type enhancers generally will have a molecular weight of greater than 150 but less than 1000. In addition, the plasticizer-type enhancers should also be relatively water insoluble or they will leach into the subcutaneous tissue layers below the stratum corneum. Thus, plasticizer-type enhancers with water solubility of less than 0.5 wt % are preferred, and more preferably 0.2 wt % or less.

A preferred group of plasticizer-type enhancers includes lower alkyl and alkoxy esters of pharmaceutically acceptable fatty acids, fatty acid esters, fatty alcohols, and similar hydrophobic compounds. As used herein, the term ‘lower alkyl and lower alkoxy’ refers to alkyl and alkoxy groups-having up to and including 7 carbon atoms and preferably, up to and including 4 carbon atoms. Some examples of alkyl groups include methyl, ethyl, propyl, isopropyl, n-butyl, tertiary butyl, pentyl, isopentyl, neopentyl, hexyl, isohexyl and heptyl. Some examples of alkoxy groups include the oxides corresponding to the above alkyl groups. Examples of suitable fatty acid esters include saturated or unsaturated fatty acid esters, including isopropyl myristate, isopropyl palmitate, and the methyl and ethyl esters of oleic and lauric acid. Suitable fatty alcohols include stearyl alcohol and oleyl alcohol. Examples of suitable fatty acids include saturated and unsaturated fatty acids, including oleic acid, lauric acid, myristic acid, palmitic acid, stearic acid, linoleic acid, and palmitoleic acid. In addition, many other compounds can also serve as plasticizer-type enhancers, such as diethyl hexyl phthalate, octyldocecyl myristate, isostearyl isostearate, caprylic/capric triglycerides including polyethylene glycol esters of caprylic/capric acids, propylene glycol laurate (Lauroglycol), Miglyol (propylene glycol diester caproic, caprylic, capric, lauric acid), Lexol PG-865 (propylene glycol diester decanoic, octanoic acid), propylene glycol myristate (mirpyl), corn oil polyethylene glycol-6 esters (Labrafil M2124CS), polyethylene glycol-8 caprylic capric glycerides (Labrasol), caprylic/capric triglycerides (Labrafac Lipophile WL 1349), caprylic/capric triglyceride polyethylene glycol-4 esters (Labrafac Hydro WL1219 available from Gattefosse, Westwood N.J.), glyceryl oleate, hexamethyldisiloxane, m dimethicone, cyclomethicone, squalene, mineral oil, macrocyclic ketones/lactones, plant extracts such as Chrodarom Calendula O or Chrodarom Chamomile O, and various oils including wintergreen, jojoba oil, or eucalyptol (cineole). A preferred plasticizer-type enhancer for use with the mesylate salt of Rec 15/2739 include caprylic/capric acids triglyceride PEG-4 esters, available as Labrafac Hydro WL 1219, (Gattefosse, Westwood, N.J.) which contains a mixture of saturated polyglycolyzed glycerides consisting of glycerides and polyethylene glycol esters of caprylic and capric acids. One of skill in the art would appreciate that the plasticizer-type enhancers may be used alone or in combination. A particularly preferred enhancer combination including the caprylic/capric triglycerides for use with the mesylate salt of Rec 15/2739 is an enhancer vehicle consisting essentially of ethanol:caprylic/capric triglycerides polyethylene glycol-4 ester:propylene glycol:isopropyl myristate in a 1:1:1:1 ratio.

As used herein, “solvent-type enhancer” generally refers to relatively hydrophilic compounds having molecular weights of less than about 200 that are capable of increasing the permeability of Actives to the stratum comeum. Solvent-type enhancers typically exhibit solubility parameters between about 10 and 24, and preferably between about 10 and 18. Solvent-type enhancers are often better enhancers because they generally provide higher flux rates for a given permeant than plasticizer-type enhancers. Typically, the solvent type enhancers will comprise a pharmaceutically-acceptable lower alkyl alcohol, aryl alcohol, or polyol, for example, ethanol, propanol, butanol, benzyl alcohol, glycerin, or propylene glycol. In some embodiments, the solvent-type enhancer is a 2-pyrrolidone or alkyl derivative thereof, such as N-methyl-2-pyrrolidone, 3-hydroxy-N-methyl-2-pyrrolidone, and pyroglutamic acid esters.

Other embodiments may employ an alkyl ether, such as ethylene, polyethylene or propylene glycol ether, as the solvent type enhancer. Preferred examples of ethylene glycol ethers include, but are not limited to, ethylene glycol monoalkyl ethers, such as ethylene glycol monomethyl ether (also known as methyl cellosolve), ethylene glycol dialkyl ethers, such as ethylene glycol dimethyl ether (also known as dimethyl cellosolve), and ethylene glycol monoalkyl ether esters, such as ethylene glycol monoethyl ether acetate (also known as cellosolve acetate). Preferred examples of polyethylene glycol ethers include, but are not limited to, diethylene glycol monoalkyl ethers, such as diethylene glycol monobutyl ether (also known as butyl ethyl Cellosolve or butyl carbitol), diethylene glycol dialkyl ethers; and diethylene glycol monoalkyl ether esters, such as diethylene glycol monoethyl ether acetate (also known as Carbitol acetate), and transcutol (diethylene glycol monoethyl ether).

Preferred solvent type enhancers have a molecular weight of less than about 150. They are also relatively hydrophilic, generally being greater than 2 wt % soluble in water, and are preferably greater than 10 wt % soluble in water. Most preferred solvent type enhancers are completely water miscible. One of skill in the art would appreciate that the solvent type enhancers may be used alone or in combination.

The percentage by weight of the Active in the solution, hydrogel or matrix may be varied according to the desired loading of the finished patch.

Compositions of the present invention are administered as indicated above, preferably once or twice or more often per day, as needed, to provide the suggested dietary supplement. More preferably, the composition comprises the following ingredients that is taken as a nutritional supplement one to five times per day.

In a particularly preferred aspect of the present invention, the transdermal delivery system may be designed to delivery parthenolide at the indicated rate for an extended period. Thus, preferably the transdermal delivery system will administer parthenolide at the indicated rates for periods of 24 hours, or from about 1 to about 3 days, about 3 to about 7 days for from about 1 week to about 4 weeks. Longer rates of delivery of parthenolide are also contemplated. The extended controlled dose delivery of parthenolide as described herein provide particular benefit to the user wishing to have convenient administration of a supplemental amount of parthenolide without daily administration. Transdermal delivery systems capable of delivering an effective dose of parthenolide over a period of about 3-7 days are particularly advantageous for periods in which an elevated amount of parthenolide supplementation is desired, such as during menses.

Dietary supplements of the present invention may have beneficial effects on the health and well being of patients particularly due to the biological impact that parthenolide and other sesquiterpene lactones, especially those containing an α-methylene-γ-lactone group, have demonstrated. Specifically, these compounds have been shown to possess activity against tumor growth and general inflammation. Specific sesquiterpene lactones have been asserted to have desired effects on the Nuclear factor (NF)-kB pathway, which is intimately involved in many disease states. The NF-kB pathway was first discovered in 1986. The pathway is central and is essential for basic immune response. It is also intimately involved in many disease states, including those of chronic inflammation (arthritis, inflammatory bowel disease, multiple sclerosis, etc.), those of acute, intermittent inflammation (migraine, asthma, etc.), those of auto-immune disease (lupus, fibromyalgia, autoimmune myocarditis, etc.), those of dysfunctional immune response (cancer, AIDS, etc.), those associated with infectious agents and parasites (Hepatitis B&C,H. pylori, malaria, tuberculosis, etc.), those associated with endocrine function (diabetes, pancreatitis, etc.) those associated with degenerative proceses (aging, alzheimers, etc.) those genetically mediated (muscular dystrophy, etc.), and those associated with trauma (heat shock, post-perfusion injury, restenosis after angioplasty, etc.) among many others.

Specific genes known to be regulated by NF-KB and implicated in disease include: Cyclin D1 (cancer); IL-8 (asthma); MCP1 (atherosclerosis); MMP9 (cancer, arthritis); c-Myc (cancer); 5′ deiodinase (euthyroid sick syndrome); HIV LTR (AIDS); Bc1-xL (cancer); c-LAP2 (cancer); iNOS (septic shock); COX-2 (inflammation, colorectal cancer).

All patents, patent documents, and publications cited herein are incorporated by reference as if individually incorporated. Unless otherwise indicated, all parts and percentages are by weight. The foregoing detailed description has been given for clarity of understanding only. It will be appreciated that numerous modifications and variations of the invention are possible in light of the above teachings, and therefore the invention may be practiced otherwise than as particularly described.

What is claimed is:

1. A method of administering a dietary supplement comprising parthenolide, comprising a) providing a transdermal dosage composition comprising a dietary supplemental amount of parthenolide in a predetermined dosage amount of at least about 1 mg of parthenolide; and
b) transdermally administering the dosage composition to a patient.

2. The method of claim 1, wherein the composition comprises sufficient parthenolide to transdermally administer from about 1 to about 50 mg of parthenolide.

3. The method of claim 1, wherein the composition comprises sufficient parthenolide to transdermally administer from about 1 to about 30 mg of parthenolide.

4. The method of claim 1, wherein said composition additionally comprises ginger extract.

5. The method of claim 1, wherein said composition additionally comprises green tea extract.

6. The method of claim 1, wherein said composition comprises additional sesquiterpene lactones.

7. The method of claim 1, wherein said composition contains substantially no active ingredients other than those that are extractable from herbal sources.

8. The method of claim 1, wherein said composition contains substantially no active ingredients other than those that are extractable from feverfew, ginger and green tea sources.

9. The method of claim 7, wherein said composition contains less than about 400 mg of any given natural active ingredient per dose.

10. A topically applied composition for transdermally delivering parthenolide to a patient in need thereof, said composition comprising parthenolide in an amount sufficient to administer to the patient a total transdermally administered amount of from about 1 mg to about 50 mg of parthenolide within a two hour period.

11. A topically applied composition for transdermally delivering parthenolide to a patient in need thereof, said composition comprising parthenolide in an amount sufficient to administer to the patient a total transdermally administered amount of from about 1 mg to about 50 mg of parthenolide in a 24 hour period.

12. The composition of claim 10, said composition being a lotion.

13. A transdermal delivery patch comprising parthenolide, said patch being engineered to deliver parthenolide to a patient transdermally at a rate of about 1 mg to about 50 mg of parthenolide in a two hour period.

14. The transdermal delivery patch of claim 13, said patch comprising a backing layer and an adhesive matrix layer comprising parthenolide.

15. The transdermal delivery patch of claim 13, said patch comprising a backing layer, a matrix layer comprising parthenolide on one surface of said backing layer, and a peripheral adhesive layer positioned to adhere the patch to a patient while allowing contact of at least a portion of the matrix layer to the patient.

16. The transdermal delivery patch of claim 13, said patch comprising a backing layer, a reservoir comprising parthenolide on said backing layer, an inert membrane on the opposite side of the reservoir from the backing layer, and an adhesive layer positioned to adhere the patch to a patient.

17. The patch of claim 13, wherein the inert membrane is a rate controlling membrane for regulating the rate of transmission of parthenolide from the patch to be transdermally administered to the patient.

18. The patch of claim 13, wherein the adhesive layer completely covers one surface of the inert membrane, so that parthenolide migrates through the adhesive layer to be transdermally administered to the patient.

19. A transdermal delivery patch comprising parthenolide, said patch being engineered to deliver parthenolide to a patient transdermally at a rate about 1 mg to about 50 mg of parthenolide in a 24 hour period.
